(19) United States
(12) Patent Application Publication
(10) Pub. No.: US 2021/0330960 A1
(43) Pub. Date: Oct. 28, 2021

(54) PIVOT LATCH MECHANISM FOR EXTERNAL BATTERY

(71) Applicant: HeartWare, Inc., Miami Lakes, FL (US)

(72) Inventors: Sacha Hall, Westborough, MA (US); Thai Nguyen, Minneapolis, MN (US)

(21) Appl. No.: 17/149,256

(22) Filed: Jan. 14, 2021

Related U.S. Application Data
(60) Provisional application No. 63/014,165, filed on Apr. 23, 2020.

(51) Int. Cl. A61M 60/876; A61M 60/81; A61M 60/878; A61M 60/122

(52) U.S. Cl. CPC A61M 60/876; A61M 60/81; A61M 2205/04; A61M 60/122; A61M 60/878

(57) ABSTRACT

An attachment mechanism between a controller for an implantable blood pump and a battery housing. The attachment mechanism includes a latch moveably coupled to the controller. The latch includes a pawl configured to engage the battery housing. The latch is configured to only pivot when the battery housing is engaged to the pawl during attachment of the battery housing to the controller.

### CROSS-REFERENCE TO RELATED APPLICATION

This application claims the benefit of U.S. Application Ser. No. 63/014,165, filed Apr. 23, 2020.

### FIELD

The present technology is generally related to attachment mechanisms for controllers and batteries of implantable medical devices, and in particular, controllers and batteries for implantable blood pumps.

### BACKGROUND

External controllers for implantable blood pumps not only control, at least in part, operation of the implantable blood pump, but also connect with a battery which supplies power to the controller and the implantable blood pump. The batteries provide a certain charge time, for example, a few hours, and then are recharged and recoupled to the controller. Thus, the user repeatedly attaches and detaches batteries to the controller, which may increase the prevalence of the controller and battery getting jammed together and being unable to attach or detach.

### SUMMARY

The techniques of this disclosure generally relate to attachment mechanisms for controllers and batteries of implantable medical devices, and in particular, controllers and batteries for implantable blood pumps.

In one aspect, the present disclosure provides an attachment mechanism between a controller for an implantable blood pump and a battery housing. The attachment mechanism includes a latch moveably coupled to the controller. The latch includes a pawl configured to engage the battery housing. The latch is configured to only pivot when the battery housing is engaged to the pawl during attachment of the battery housing to the controller.

In another aspect of this embodiment, the controller includes at least one pivot slot configured to engaged at least one corresponding pivot tab on the battery housing.

In another aspect of this embodiment, the latch is configured to only pivot when the battery housing is disengaged from the pawl during detachment of the battery from the controller.

In another aspect of this embodiment, the latch is coupled to a spring.

In another aspect of this embodiment, the latch includes an actuator, and wherein the actuator is movable.

In another aspect of this embodiment, the spring is axially aligned within pawl and substantially perpendicular to the actuator.

In another aspect of this embodiment, the latch pivots about a pin.

In another aspect of this embodiment, the pawl includes a projection at its distal end.

In another aspect of this embodiment, when the battery housing is attached to the controller, the controller is in electrical communication with a battery within the battery housing.

In one aspect, an attachment mechanism between a controller for an implantable blood pump and a battery housing includes a latch moveably coupled to the controller. A strike plate is coupled to the battery housing. The latch includes a pawl configured to engage the strike plate. The latch is configured to only pivot when the strike plate is engaged to the pawl during attachment of the battery housing to the controller.

In another aspect of this embodiment, the controller includes at least one pivot slot configured to engage at least one corresponding pivot tab on the battery housing.

In another aspect of this embodiment, the latch is configured to only pivot when the strike plate is disengaged from the pawl during detachment of the battery housing from the controller.

In another aspect of this embodiment, the latch is coupled to a spring.

In another aspect of this embodiment, the latch includes an actuator, and wherein the actuator is movable.

In another aspect of this embodiment, the spring is axially aligned within pawl and substantially perpendicular to the actuator.

In another aspect of this embodiment, the latch pivots about a pin.

In another aspect of this embodiment, the pawl includes a projection at its distal end.

In another aspect of this embodiment, the projection engages the strike plate.

In another aspect of this embodiment, when the battery housing is attached to the controller, the controller is in electrical communication with a battery within the battery housing.

In one aspect, an attachment mechanism between a controller for an implantable blood pump and a battery housing includes a latch moveably coupled to the controller. A strike plate is coupled to the battery housing. The controller includes a pair of pivot slots configured to engage a pair of corresponding pivot tabs on the battery housing. The latch includes a pawl configured to engage the strike plate. The latch is configured to only pivot when the strike plate is engaged to the pawl during attachment of the battery housing to the controller. The latch is configured to only pivot when the strike plate is disengaged from the pawl during detachment of the battery housing from the controller.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a front perspective view of a controller with a latch and a battery housing constructed in accordance with the principles of the present application;

FIG. 2 is a cross-sectional view of the controller with a latch and battery housing shown in FIG. 1;

FIG. 3 is a side view of the controller shown in FIG. 1;

FIG. 4 is a side view of the battery housing showing in FIG. 1;

FIG. 5 is an exploded view of the latch shown in FIG. 1 within the controller;

FIG. 6 is an assembled view of the latch shown in FIG. 1 within the controller;

FIG. 7 is a side cross-sectional view showing the latch shown in FIG. 1 pivoting to attach and detach the battery housing;

FIG. 8 is an exploded view of another latch constructed in accordance with principles of the present application with the controller;

FIG. 9 is an assembled view of the latch shown in FIG. 8;

FIG. 10 is side cross-sectional view of the latch shown in FIG. 8 pivoting to attach the battery housing; and

FIG. 11 is side cross-sectional view of the latch shown in FIG. 8 translating to detach the battery housing.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

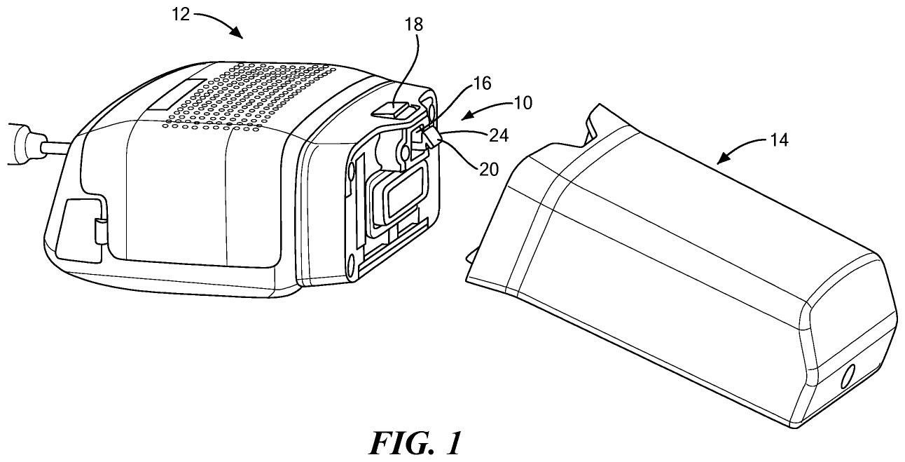

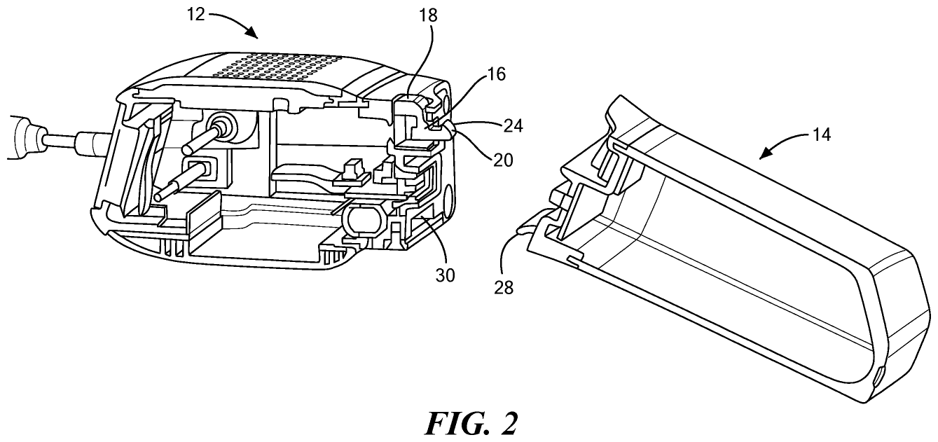

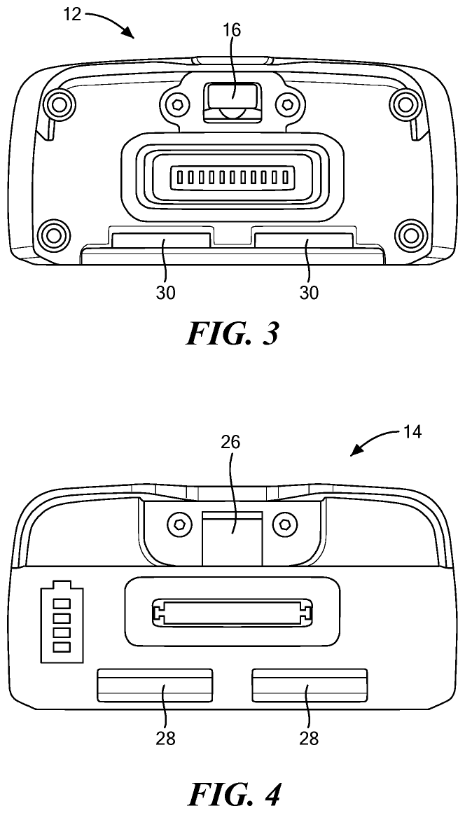

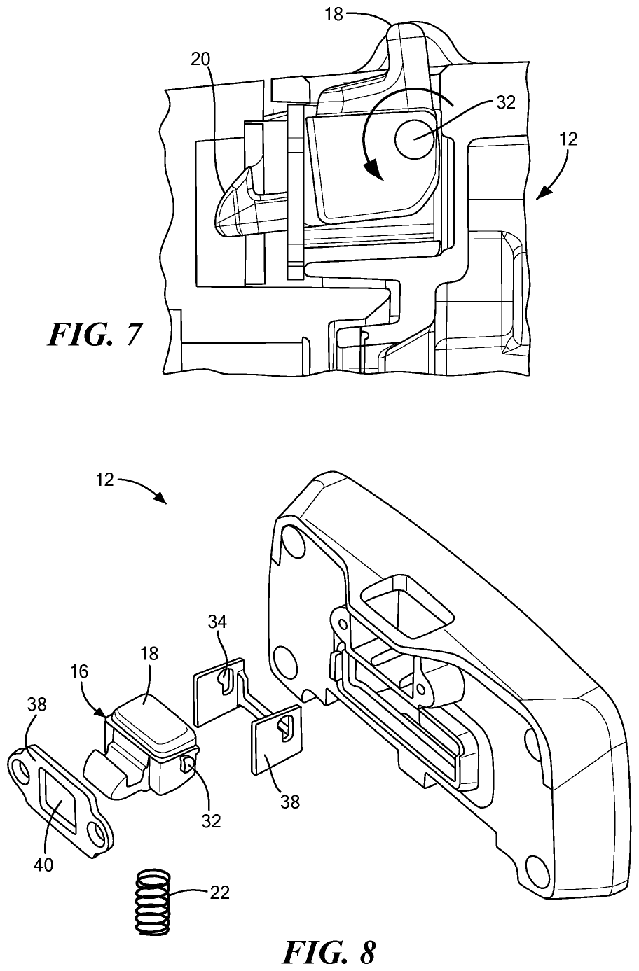

Referring now to the drawings in which like reference designators refer to like elements there is shown in FIGS. 1-7 and exemplary attachment mechanism between a controller and a battery housing and designated generally as “10.” The attachment mechanism 10 is configured to releasably connect a controller 12 for a medical device, such as an implantable blood pump, with a battery within a battery housing 14. When connected the battery provides power to the controller 12 for operating the blood pump among other functions. The attachment mechanism 10 includes a latch 16 disposed at a distal end of the controller 12 configured to releasably attach with a proximal end of the battery housing 14. The latch 16 includes an actuator 18, a pawl 20 extending outwardly from the latch 16 away from the controller 12, and a biasing element 22, such as a spring. In one configuration, the biasing element 22 is axially aligned with the pawl 20 and substantially orthogonally to the actuator 18. The pawl 20 includes a projection 24 at its distal end configured to grasp a portion of the battery housing 14. In particular, the battery housing 14 includes a strike plate 26 configured to engage the pawl 20 and its projection 24. The battery housing includes at least one pivot tab 28, for example, two pivot tabs 28, extending away from the battery housing 14 and spaced a distance from the strike plate 26. The at least one pivot tab 28 is configured to engage a corresponding at least one pivot slot 30, for example, two pivot slots 30 on the controller 12, to align the controller 12 with the battery housing 14. Once the pivot tabs 28 are engaged to the pivot slots 30, the user can pivot the battery housing 14 with respect to the controller 12 to engage the strike plate 26 with the pawl 20.

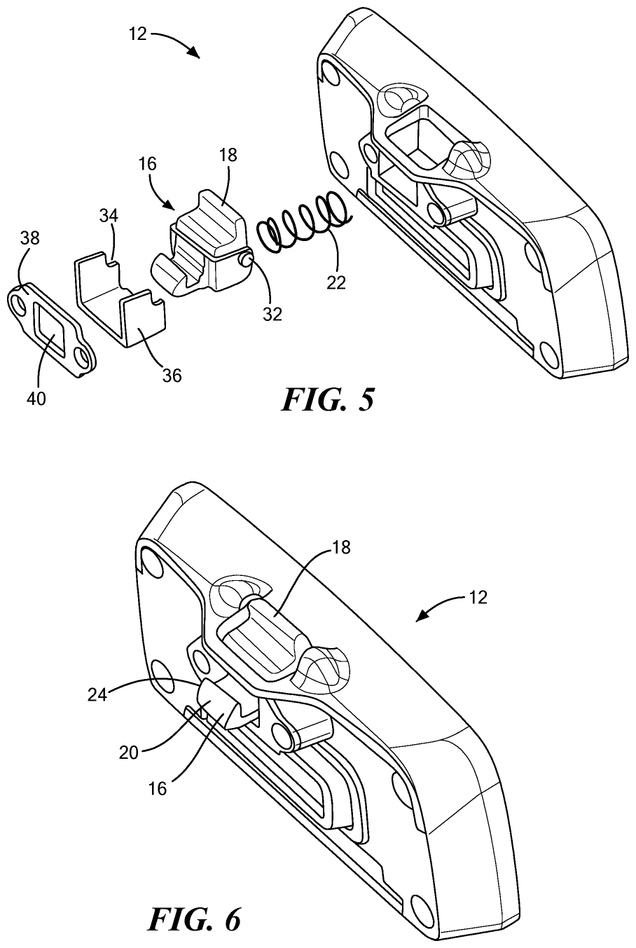

Referring now to FIGS. 5-7, the latch 16 is configured to only pivot when the battery housing 14 is engaged to the pawl 20 during attachment of the battery housing 14 to the controller 12. For example, as discussed above, when the strike plate 26 engages the projection 24 of pawl 20, it pushes on the latch 16 against the biasing member 22 such that it pivots toward the controller 12. As the user continues to push the strike plate 26 against the pawl 20 and further pushes the biasing element 22, the latch 16 continues to pivot until the strike plate 26 is seated within the pawl 20, at which point the biasing member 22 pushes on the latch 16 and causes it to pivot in the opposite direction and lock the battery housing 14 to the controller 12. To detach the battery housing 14 from the controller 12, the user pushes on the actuator 18 in the direction of the pawl 20, which causes the latch 16 to pivot toward the controller 12 and releases the strike plate 26 from the pawl 20. The latch 16 includes a pin 32 extending outward therefrom. The pin 32 is configured to be received within a corresponding slot 34 defined by a retainer 36. In particular, the latch 16 is pivotally engaged within the retainer 36 and the pin 32 moves within the slot 34 as the user pushes on the actuator 18. A plate 38 having window 40 is coupled to the controller 12 and affixes both the latch 16 and the retainer 36 to the controller 12. The pawl 20 protrudes from the window 40 such that the pawl is accessible by the strike plate 26.

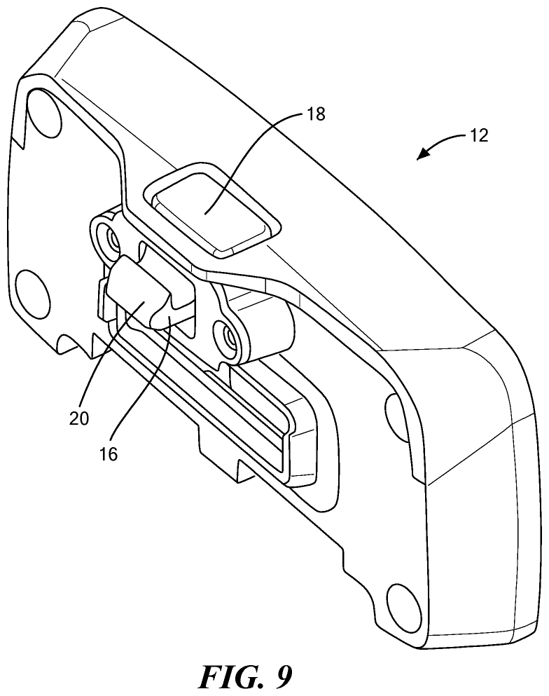

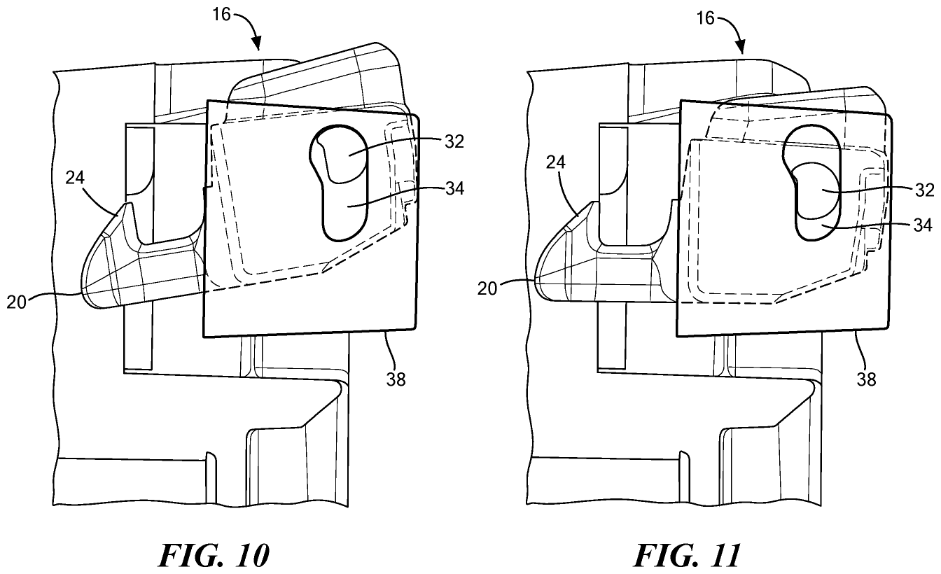

In another configuration, the biasing element 22 is perpendicular to the pawl 20 and the pin 32 is configured to translate longitudinally within the slot 34. For example, as shown in FIGS. 8-11, the pin 32 is configured to only pivot within slot 34 when the strike plate 26 is engaged to the pawl 20 when the battery housing 14 is attached to the controller 12. To release the battery housing 14 from the controller, the user presses on the actuator 18 which causes the latch 16 to only translate downward and moves the pin 32 downward within the slot 34 and releases the battery housing 12 from the controller 12.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An attachment mechanism between a controller for an implantable blood pump and a battery housing, comprising:
a latch moveably coupled to the controller; and
the latch including a pawl configured to engage the battery housing, the latch being configured to only pivot when the battery housing is engaged to the pawl during attachment of the battery housing to the controller.

2. The mechanism of claim 1, wherein the controller includes at least one pivot slot configured to engaged at least one corresponding pivot tab on the battery housing.

3. The mechanism of claim 1, wherein the latch is configured to only pivot when the battery housing is disengaged from the pawl during detachment of the battery from the controller.

4. The mechanism of claim 1, wherein the latch is coupled to a spring.

5. The mechanism of claim 4, wherein the latch includes an actuator, and wherein the actuator is movable.

6. The mechanism of claim 5, wherein the spring is axially aligned within pawl and substantially perpendicular to the actuator.

7. The mechanism of claim 6, wherein the latch pivots about a pin.

8. The mechanism of claim 1, wherein the pawl includes a projection at its distal end.

9. The mechanism of claim 1, wherein when the battery housing is attached to the controller, the controller is in electrical communication with a battery within the battery housing.

10. An attachment mechanism between a controller for an implantable blood pump and a battery housing, comprising:
a latch moveably coupled to the controller;
a strike plate coupled to the battery housing; and
the latch including a pawl configured to engage the strike plate, the latch being configured to only pivot when the strike plate is engaged to the pawl during attachment of the battery housing to the controller.

11. The mechanism of claim 10, wherein the controller includes at least one pivot slot configured to engage at least one corresponding pivot tab on the battery housing.

12. The mechanism of claim 10, wherein the latch is configured to only pivot when the strike plate is disengaged from the pawl during detachment of the battery housing from the controller.

13. The mechanism of claim 10, wherein the latch is coupled to a spring.

14. The mechanism of claim 13, wherein the latch includes an actuator, and wherein the actuator is movable.

15. The mechanism of claim 14, wherein the spring is axially aligned within pawl and substantially perpendicular to the actuator.

16. The mechanism of claim 15, wherein the latch pivots about a pin.

17. The mechanism of claim 10, wherein the pawl includes a projection at its distal end.

18. The mechanism of claim 17, wherein the projection engages the strike plate.

19. The mechanism of claim 10, wherein when the battery housing is attached to the controller, the controller is in electrical communication with a battery within the battery housing.

20. An attachment mechanism between a controller for an implantable blood pump and a battery housing, comprising:
a latch moveably coupled to the controller;
a strike plate coupled to the battery housing;
the controller includes a pair of pivot slots configured to engage a pair of corresponding pivot tabs on the battery housing;
the latch including a pawl configured to engage the strike plate, the latch being configured to only pivot when the strike plate is engaged to the pawl during attachment of the battery housing to the controller; and
the latch is configured to only pivot when the strike plate is disengaged from the pawl during detachment of the battery housing from the controller.
